(19) United States
(12) Patent Application Publication
(10) Pub. No.: US 2026/0063651 A1
(43) Pub. Date: Mar. 5, 2026

(54) APPARATUS FOR REAL-TIME MONITORING OF ACTIVATED CLOTTING TIME AND METHOD OF USE

(71) Applicant: Amit N. Amin, Lafayette, LA (US)

(72) Inventor: Amit N. Amin, Lafayette, LA (US)

(21) Appl. No.: 19/313,863

(22) Filed: Aug. 29, 2025

Related U.S. Application Data
(60) Provisional application No. 63/688,497, filed on Aug. 29, 2024.

(51) Int. Cl. A61B 5/15; A61B 5/00; A61B 5/0535; G01N 33/86

(52) U.S. Cl. CPC G01N 33/86

(57) ABSTRACT

An activated clotting time measurement apparatus comprising a catheter having first and second lumens inserted into a vascular vessel of a patient and a sensor module. The first and second lumens are connected to a peristaltic pump whereby aliquots of blood is collected from the patient's vascular vessel and delivered to a sensor cartridge in the sensing module by the first lumen via a control valve and uncollected blood is returned to the patient's vascular vessel in the second lumen. The sensor cartridge has a sensing channel, a pair of electrodes, and a clotting activator whereby the aliquots of blood are clotted. A current generator generates current to the electrodes through the clotted blook aliquots where an impedance measurement device measures the electrical impedance caused by the clotted blood and delivers impedance signals to a processor and display device for processing and display.

### FIELD OF THE INVENTION

This invention relates to a device and method for real time testing and display of activated clotting time test result. More particularly, the invention relates to an apparatus and method for real time testing of periodic blood samples obtained from an indwelling venous catheter, measuring the activated clotting time of such blood samples, and displaying the measurement results at the patient's bedside.

### BACKGROUND OF THE INVENTION

An activated clotting time test, referred to herein as an ACT, is a laboratory test used to determine the anticoagulant effect of anticoagulation medications, such as warfarin and heparin. An ACT measures the time it takes for a blood sample to clot after a specific clotting activator is added to the blood sample. A number of clotting activator are typically used including celite, kaolin, or ellagic acid.

Anticoagulation medications and therapies are used during medical procedures, including cardiac catheterization, angioplasty, bypass surgery, dialysis, and for a variety of medical conditions. Such medical conditions include, but are not limited to, cardiovascular diseases, deep vein thrombosis, and pulmonary embolism. ACT measurement is crucial for monitoring the effectiveness and safety of such anticoagulation medications and therapies.

When cardiac and cardiovascular procedures are undertaken, an ACT is determined after an initial dose of anticoagulant medication is administered pre-operatively and before such procedures are started. During cardiac and cardiovascular procedures, an ACT is obtained at regular intervals in order to determine whether the effect of the anticoagulation is being maintained at a desired level. After such procedures, ACT is monitored until the patient is stable or until the ACT reaches a desired level. ACT may also be obtained when a patient has a bleeding episode, or to evaluate a patient's response to anticoagulation level, or when a patient's blood coagulation is in question due to coagulation factor deficiency, severe thrombocytopenia, or severe platelet dysfunction.

ACT test results must be obtained rapidly as they are needed to manage the ongoing treatment. ACT is measured in seconds and the ACT test must be performed immediately after the blood is collected from a patient, usually a site located near the bedside, in the operating room, or in a satellite laboratory close to these locations. For testing, a discrete whole blood specimen is collected, commonly between 0.5 to 1 mL of blood, from an indwelling or extracorporeal line in a patient's blood vessel. The collected specimen is collected into a syringe or other tube and immediately transferred into a testing device cartridge or cuvette. The ACT test should be completed no more than 1 to 2 minutes after the blood sample is collected and the blood temperature should be maintained at a constant temperature, normally 37° C., in order to maintain the accuracy of the test. When periodic ACT is required such as during cardiovascular procedures and surgery, multiple discrete samples of blood must be obtained.

Existing methods of measuring ACT require larger blood samples which are often obtained through venipuncture and transferred by hand to a testing device. Obtaining such samples can be cumbersome, time-consuming, and uncomfortable for patients. Changes in the blood sample temperature after its collection and during its testing may also affect ACT results. Inaccuracies in determining ACT remain evident in current testing modalities which can compromise patient care and result in patient harm and life threatening complications.

Accordingly, there is a need for an ACT testing apparatus and method that will eliminate the need for obtaining discrete samples of whole blood for testing.

There is also need for an ACT testing apparatus and method that will provide continuous ACT results during surgical and cardiovascular procedures.

The is also a need for an ACT testing apparatus and method that will eliminate testing errors resulting from the manual collection and transfer of blood testing to testing devices.

### SUMMARY OF THE INVENTION

The present invention provides an apparatus and method for real time ACT testing to satisfy the aforementioned needs and to overcome the limitations of existing ACT measurement methods. The apparatus and method allow for the collection of micro aliquots of blood, microliters rather than milliliters, from an aspiration catheter placed within an indwelling venous catheter. An impedance sensor in combination with the aspiration catheter then analyzes the micro aliquots of blood so collected to provide in real time ACT results which are delivered to the healthcare professionals on a digital display.

The apparatus and method of the present invention allow for efficient and accurate measurement of clotting time, enabling timely monitoring and management of patients receiving anticoagulant therapy. When employed, the apparatus and method will deliver blood samples for ACT testing and ACT results more conveniently and efficiently while minimizing patient discomfort and with less compromised patient care.

Because only microliter blood samples are extracted, and at predetermined times, there is a significant reduction. As a result, patient safety will be increased due to the reduction in blood needed for required ACT testing. There will also be a corresponding reduction in the time required for cardiac and endovascular procedures, a decrease in the amount of anticoagulation medications, such as warfarin and heparin, used in such procedures, and an overall decrease costs.

The apparatus is comprised of a catheter inserted in a patient's venous blood vessel, through an introducer sheath and a sensor module. The inserted catheter has two lumens. The first lumen allows blood to be removed from the vessel to a peristaltic pump. The second lumen delivers unconsumed blood from the peristaltic pump back to the patient's vessel. An electrically activated control entry valve in the first lumen provides an entry way for aliquots of the circulating blood to be delivered into the sensor module. The peristaltic pump maintains a continuous flow of fresh blood throughout the catheter lumens.

The sensor module is comprised of a measurement subsystem having a microfluidic cartridge. The microfluidic cartridge is provided with at least one sensing channel or cell having impedance testing electrodes. At least one wall of the sensing channel or cell is provided with an activator coating such a silica to trigger a blood clotting cascade. A syringe pump is provided for delivering aliquots of blood to the sensing channel. The microfluidic cartridge is also provided with resistive heating elements as a heat source to maintain the blood temperature to be maintained at physiological levels (37° C.) throughout the testing period. Multiple sensing channels or cells may be provided to allow for staged parallel testing.

The impedance testing electrodes are made of a conductive material conducive to bioelectrical measurement of blood. Suitable impedance testing electrodes may include gold and silver-silver chloride electrodes. The impedance testing electrodes come into direct contact with fresh blood to measure the blood's electrical impedance. The measurement subsystem has a power supply configured to generate a DC electrical current across the impedance testing electrodes through the aliquot of blood in the sensing channel. Because blood can act as a complex impedance, the impedance measurement subsystem is configured to generate a known excitation voltage at a particular frequency ranging from 1 kOhm to 100 kOhm. Impedance measurement occurs continuously through the aliquot of blood in the sensing channel, across the two electrodes.

Control sensors and actuators in communication with a microprocessor and microcontroller and associated drivers control the operations of the testing apparatus. The microprocessor and microcontroller perform real-time control of the blood aliquots, blood temperature, testing current, the testing computations, and their precise timing. The microprocessor and microcontroller then deliver control signals to the sensor and actuators to control the testing operations and deliver testing status and measurement data to a storage device and to a display monitor. The display monitor allows the testing status and measurement data to be readily available in real time for patient care by healthcare professionals.

### BRIEF DESCRIPTION OF DRAWINGS

FIG. 1 is a block diagram of the components of the real time ACT testing apparatus of the present invention.

FIG. 2 is a schematic view of the catheter, insertion sheath, and catheter insertion of the invention shown in FIG. 1.

FIG. 3 is an exploded view of the catheter and catheter components of the catheter shown in FIG. 2

FIG. 4 is a schematic cross-section view of a sensing channel of the sensing cartridge of the invention shown in FIG. 1 showing an activator placement device.

FIG. 5 is a schematic view of an alternate embodiment of the sensing cartridge of the invention shown in FIG. 1 having multiple sensing channels each with an activator placement device.

FIG. 6 is a schematic view of an alternate embodiment of the sensing cartridge of the invention shown in FIG. 1 having multiple sensing channels each with an activator placement device.

FIG. 7 is schematic view of still another alternate embodiment of the sensing cartridge of the invention shown in FIG. 1 having multiple sensing channels each with an activator placement device.

FIG. 8 a diagram showing the method steps for using the real time ACT testing apparatus of FIG. 1.

### DESCRIPTION OF THE INVENTION

The present invention is an apparatus and method for real time ACT testing intended to alleviate the limitations of existing ACT measurement devices and methods. The ACT testing apparatus (100) of the present invention and its method of use allow for the collection and testing of microliter blood samples drawn directly from a patient's vascular system by an indwelling catheter while providing immediate display of the ACT testing results.

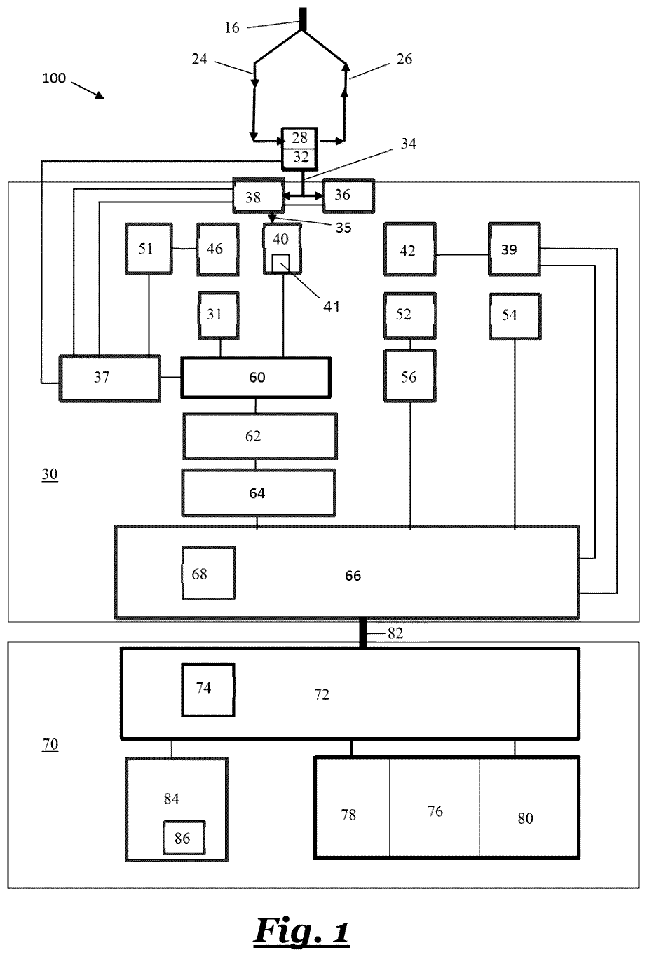

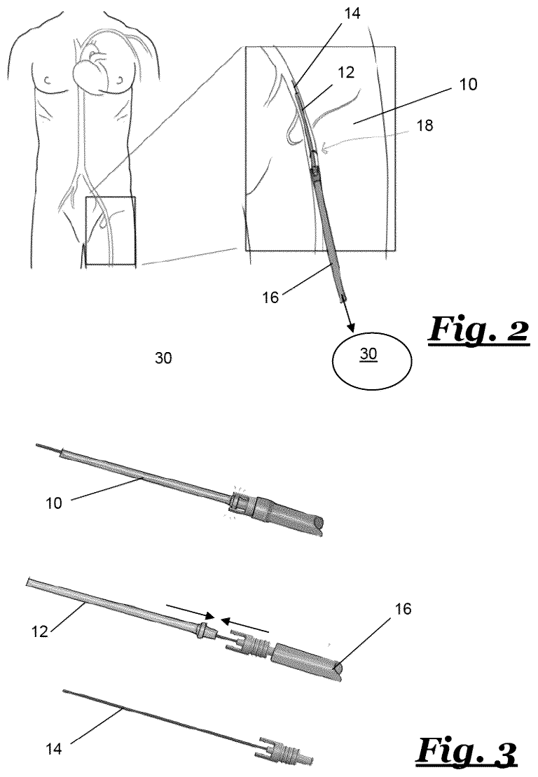

As shown in FIGS. 1-3, the apparatus 100 is comprised of a catheter 10 that is inserted into a patient's venous blood vessel 18. Catheter 10 is comprised on an introducer sheath 12, a catheter probe 14, and a blood supply tube 16. Supply tube 16 of the catheter 10 has two lumens. The first supply tube lumen 24 allows blood 20 to be removed from the catheter supply tube 16 by a peristaltic pump 28. The second supply tube lumen 26 recirculates unconsumed blood 20 from the peristaltic pump 28 back to the patient's vessel. The peristaltic pump 28 maintains a continuous flow of fresh blood 20 through the supply tube lumens 24 and 26 of the catheter 10 for delivery to a sensor module 30. The continuous flow 20 through the supply tube lumens 24 and 26 minimizes the probability of clot formation in the fresh blood 20.

The sensor module 30 is comprised of an electrically activated control entry valve 32 that is in fluid communication with the continuous flow of fresh blood 20 in the first supply tube lumen 24 and a blood sample lumen 34. A syringe pump 36 is configured to pull blood sample aliquots 22 of the circulating blood 20 from the blood sample lumen 34 to channel lumen 35 and, in conjunction with an array of electronically control valves 38 and pressure sensors 39, delivers the blood sample aliquots 22 to a sensor cartridge 40 containing one or more sensing electrode cells or channels 41. Pulling circulating blood 20 from the blood sample lumen 34 to channel lumen 35 shortens the path between fresh blood access and the sensing channels 41 reduces the chance of clotting and ultimately the waste of blood volume needed for testing. Stepper motor drivers 37 configured for control of stepper motors actuate the valve mechanisms in the testing module 30. A new sensing cartridge 40 will be provided for each new patient.

Each sensing channel 41 of the sensing cartridge 40 is provided with at least one pair of electrodes 42. The electrodes 42 are comprised of a conductive material, such as material comprised of gold, silver, or silver-silver chloride, conducive to bioelectrical measurement of blood. The electrodes 42 come into direct contact with blood sample aliquots 22 during the measurement process. The electrodes 42 can be formed using screen printing methods to apply a metallic paste that is cured into a solid conductive surface onto a substrate. The electrodes can also be formed using selective electroless plating and electroplating.

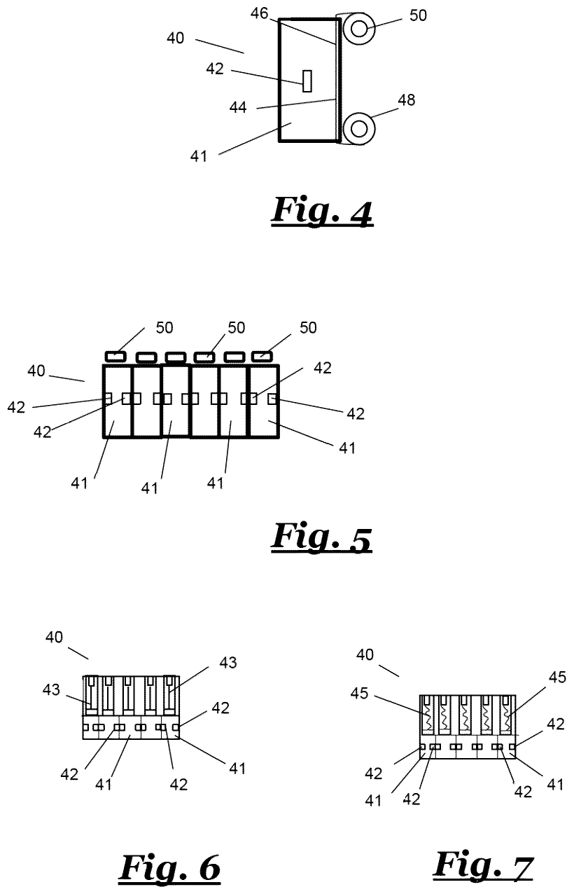

Each sensing channel 41 of the sensing cartridge 40 is further comprised of an activator 44 provided to trigger a blood clotting cascade needed to perform an ACT test. Common clotting activators include kaolin, celite, glass beads, silica and crystals thereof. In one embodiment of the sensing cartridge 40, as shown in a schematic view in FIG. 4, an activator placement device utilizes long thin plastic sheet 46 having a coating of an activator 44, such as a coating of silica, placed along one wall of the sensing channel 41. The coating of activator 44 may be applied onto the sheet 46 by developing a suspension of 6% silica in ethanol, applying the silica and ethanol solution to the plastic sheet 46, and allowing the ethanol to evaporate from the plastic sheet 46. The plastic sheet 46 with the applied activator 44 is formed into an activator roll 48 on an activator spool 50. An electronic actuator 51 rotates the spool 50 to feeds a fresh segment of sheet 46 on roll 48 with the coating of activator 44 into the sensing channel 41 of sensor cartridge 40 for each measurement cycle. Other activator placement devices may also be utilized for applying an activator 44 such as one injecting or trickling activator crystals into the sensing channel 41 during each measurement cycle by an electronically controlled linear actuator 43 as shown in FIG. 6 or by an electronically controlled screw-type conveyor actuator 45 as shown in FIG. 7.

The sensing cartridge 40 of the sensing module 30 may be comprised of any desired plurality of sensing channels 41, each having corresponding electrodes 42 and an associated activator spool 50 with an activator sheet coated with activator 44. Each sensing channel 41 may be gated by a control valve to allow for selective introduction of a fresh blood sample aliquot 22 into a selected sensing channel 41. It is thought that the sensing module 30 will perform an ACT test in approximately 2-3 minute. Providing sensing module 30 with a cartridge 40 having multiple sensing channels 41 as shown in FIG. 5 will allow for staged parallel ACT tests to be performed.

An ACT test requires the temperature of the blood sample aliquots 22 to be maintained at physiological levels (37° C.) throughout the testing period. To maintain proper testing temperatures, the testing module 30 includes a temperature sensor array 52, a heating element array 54, and a current driver 56. The heating element array 54 is comprised of resistive heating elements as a heat source while temperature sensors in the temperature sensor array 52 provide feedback to a proportional integral-derivative (PID) control system running on the microcontroller. The microcontroller uses the feedback to affect electrical current levels output by a driver.

The sensing module 30 is also comprised of an impedance measurement system 31 that converts the physical changes occurring in the blood sample aliquot 22 from analog signals into digital signals. As blood can act as a complex impedance, the impedance measurement system 31 is configured to generate a known excitation voltage at a particular frequency ranging from 1 kOhm to 100 kOhm. Impedance measurement occurs through the blood sample aliquot 22, across the two electrodes 42. An analog multiplexer 60 coupled with a signal conditioning device 62 allows a single impedance measurement device 64 to be used for measuring multiple sensing channels via temporal interlacing.

A microcontroller 66 running real-time sensor module control firmware 68 performs real-time control and computations, controls sensors and actuators in the sensor module 30 and their precise timing, while providing status and measurement data to a processing and monitor system 70.

The processing and monitor system 70 may include an application processor 72 real-time operating system running a type of custom firmware application 74. The application processor 72 may include a customized Linux operating system, random access memory, flash storage, graphics processing unit, and connectivity controllers though other types of operating systems may be utilized. The custom firmware application 74 running on the application processor 72 communicates with the sensor module 30, assists in clotting time computations, and controls a graphical user interface 76.

The graphical use interface 76 of GUI processing and monitor system 70 may be configured to include an OLED display 78 with a capacitive touch layer 80 to give healthcare professionals and clinicians a high contrast representation of the measured test components including sample temperatures, clotting time, along with trends, status messages, and configurable device parameters. The processing and monitor system 70 may be configured for pole mounting to ease its use. The processing and monitor system 70 may also be provided with built-in surge protector for maintenance of data and patient safety.

A single cable 82 connects the sensor module 30 and processing and monitor system 70, providing power to the sensor module 30 and allowing for two-way communication between the sensor module 30 and the processing and monitor system 70.

A power supply 84 is provided to convert standard 120 volt A/C outlet power to lower level DC power for use by the system. The power supply 84 can also include a backup power source 86, such as a lead acid or lithium ion battery. The backup power source 86 will allow seamless transfer of patients to transition areas without losing key data.

The sensor cartridge 40 may be provided with multiple sensing channels as shown in FIG. 5. An array of electronically controlled control valves 38 and pressure sensors 39 may be used to a deliver a blood sample aliquot 22 to a selected sensor channel 41 of the sensor cartridge 40 in a desired sequence and the testing process described above is then repeated to determine an ACT for each blood sample aliquot 22 in each select sensor channel 41.

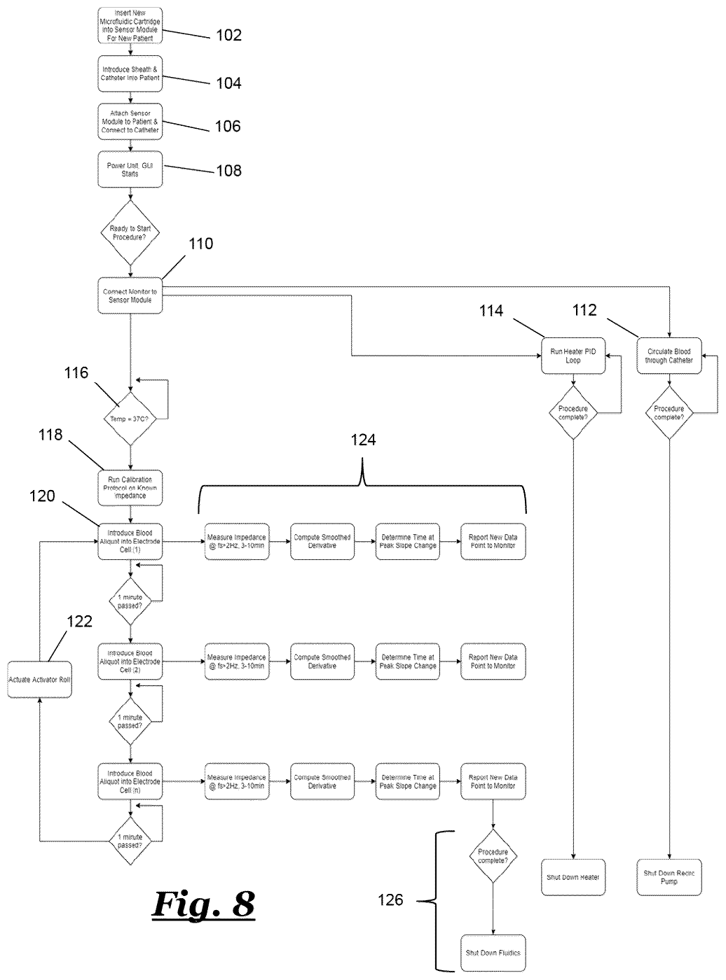

The method steps required to utilize the apparatus 100 for ACT measurement are shown in FIG. 8 and include step 102: inserting a new sensor cartridge 40 having a sensor channel 41 and an activator 44 into the sensor module 30 for a new patient; step 104: introducing the catheter 10 and introducer sheath 12 into the patient's venous blood vessel 18; step 106: attaching the catheter 10 to the sensor module 30; and step 108: starting the power supply 84 of the apparatus 100. At this point, the testing procedure is ready for starting, and, in step 110, the processing and monitor system 70 may be connected to the sensor module 30. In step 112, blood is circulated to and from the patient through catheter 10 where aliquots of blood are collected. In some embodiments the circulated blood may be circulated through a heater loop as shown in step 114.

In step 116, a selected blood aliquot 22 of the circulating blood 20 is taken from the blood sample lumen 34, delivered to the sensing module 30, where it is checked to determine whether the blood aliquot 22 is at an appropriate testing temperature, thought to be 37° C. In step 118 a calibration protocol may be run on a known impedance using the impedance measurement device 64 to verify impedance measurement.

In step 120, the selected aliquot of blood 22 is then introduced into a first electrode sensing cell or channel 41 of the sensing module 30 where in step 122 activator 44 is provided, such as by introducing activator 44 by activating activator spool 50, to trigger a blood clotting cascade. The selected aliquot of blood 22 then begins to clot over a predetermined time, preferably no more than 1 to 2 minutes after the aliquot of blood 22 is collected.

In step 124, the impedance of the aliquot of blood 22 in the first electrode cell or channel 41 is then measured over time by generating impedance signals to the processing and monitor system 70 to compute a derivative of the changes in impedance over time to determine the time of the peak clotting time which then reported on the display 78 of the processing and monitor system 70.

Providing a sensing module 30 having a sensing cartridge 40 with a plurality of sensing channels will allow for a multiple ACT test to be performed. When such additional ACT test are required, steps 116,118, 120, 122, and 124, are then repeated to obtain the additional ACT test. In step 126, when the ACT testing is completed, the recirculating peristaltic pump 28 and processing and monitor system 70 may be shut down.

The apparatus and the method described herein are merely exemplary embodiments of the invention. The apparatus and method of the present invention and many of its attendant advantages will be understood to a person of ordinary skill in the art from the foregoing description and it will be apparent that various changes may be made in the arrangement of the components of the apparatus and the method steps or the sequence of the method steps as described herein without departing from the spirit and scope of the invention or sacrificing its material advantages.

What is claimed is:

1. An activated clotting time measurement apparatus comprising:
a. a catheter having first and second lumens, said catheter configured for insertion into a vascular vessel of a patient; whereby said first lumen collects blood from said vascular vessel and whereby said second lumen returns blood to said vascular vessel;
b. a circulation pump in fluid communication with said first and second lumens, said circulation pump configured to circulate a continuous flow of blood through said first lumen and said second lumen;
c. a blood sample lumen in fluid communication with said first lumen whereby a blood sample from said continuous flow of blood is collected;
d. a sensor module having:
i. a sensing cartridge containing a sensing channel, a pair of electrodes, a clotting activator, and a channel lumen in fluid communication with said blood sample lumen;
ii. A second pump configured to pull a blood aliquot into said blood sample lumen; and
iii. a control entry valve in fluid communication with said blood sample lumen whereby said blood aliquot is delivered into said sensing channel;
e. a current generator whereby current is generated to said electrodes of said sensing channel; and
f. an impedance measurement device configured to generate impedance signals from said electrodes to a processor and display device whereby activated clotting time is calculated and displayed.

2. The activated clotting time measurement apparatus as recited in claim 1 wherein said sensor module includes resistive heating elements.

3. The activated clotting time measurement apparatus recited in claim 2 wherein said sensing cartridge is comprised of a plurality of sensing channels.

4. The activated clotting time measurement apparatus as recited in claim 3 wherein said impedance signals are transmitted as digital signals and said display is a digital display.

6. The activated clotting time measurement apparatus as recited in claim 5 wherein said sensing module is further comprised of an activator roll having a sheet upon which said clotting activator is applied.

7. The activated clotting time measurement apparatus as recited in claim 6 wherein said clotting activator is selected from the group comprising kaolin, celite, glass beads, and silica.

8. An activated clotting time measurement apparatus comprising:
a. a catheter configured for insertion into a vascular vessel of a patient, said catheter having first and second lumens;
b. a circulation pump in fluid communication with said first and second lumens, said circulation pump configured to provide a continuous flow of blood from said vascular vessel through said first lumen and return said continuous flow of blood to said vascular vessel through said second lumen;
c. a blood sample lumen in fluid communication with said first lumen;
d. a sensor module configured to receive a blood aliquot from said blood sample lumen, said sensor module having resistive heating elements, a control entry valve, a sensing cartridge containing a plurality of sensing channels in fluid communication with said blood sample lumen, each said sensing channel having a pair of electrodes and a clotting activator;
e. a current generator configured to generate current to said electrodes;
g. an impedance measurement device configured to receive impedance signals from said electrodes and deliver said impedance signals to a processor; and
h. display device whereby activated clotting time is calculated and displayed.

9. The activated clotting time measurement apparatus as recited in claim 8 wherein said sensing module is further comprised of stepper motors and stepper motor drivers configured to actuate said control entry valve.

10. The activated clotting time measurement apparatus as recited in claim 9 wherein said impedance signals are transmitted as digital signals and said display is a digital display.

11. The activated clotting time measurement apparatus as recited in claim 10 wherein said sensing module is further comprised of an activator roll having a sheet upon which said clotting activator is applied.

12. The activated clotting time measurement apparatus as recited in claim 11 further comprising pumps and control valves configured to direct said blood aliquot to a desired said sensing channel of said plurality of sensing channels.

13. The activated clotting time measurement apparatus as recited in claim 12 wherein said clotting activator is selected from the group comprising kaolin, celite, glass beads, and silica.

14. A method of continuous measurement of activated clotting time comprising the steps of:
a. Providing an activated clotting time measurement apparatus comprising:
i. a catheter having first and second lumens, said first lumen configured to collect blood from a vascular vessel and said second lumen configured to return blood to said vascular vessel;
ii. a circulation pump in fluid communication with said first and second lumens;
iii. a blood sample lumen in fluid communication with said first lumen;
iv. a sensor module having resistive heating elements, a control entry valve, a sensing cartridge containing a plurality of sensing channels in fluid communication with said blood sample lumen, each of said sensing channels having a pair of electrodes, and a clotting activator mechanism configured to introduce a clotting activator into a selected sensing channel of said a plurality of sensing channels;
v. a current generator configured to generate current to said electrodes;
vi. a plurality of pumps and control valves;
vii. an impedance measurement device; and
viii. a processor and display device configured to calculate and display activated clotting time;
b. introducing said catheter into a patient's vascular blood vessel;
c. activating said pumps and controls whereby a blood aliquot is directed from said first lumen into a said selected sensing channel;
d. activating said clotting activator mechanism to introduce said a clotting activator in said selected sensing channel thereby inducing clotting of said blood aliquot;
e. activating said current generator thereby generating current to said electrodes through said blood aliquot in said selected sensing channel;
f. generating impedance signals from said electrodes in said selected sensing channel;
g. delivering said impedance signals to said processor and display device whereby activated clotting time is calculated; and
h. displaying said calculated activated clotting time on said processor and display device.

15. The method of continuous measurement of activated clotting time as recited in claim 14 further comprising the step of measuring a known impedance with said impedance measurement device.

16. The method of continuous measurement of activated clotting time as recited in claim 15 wherein the clotting activator mechanism includes a spool having a sensor sheet roll having a coating clotting activator, and an electronic actuator configured to rotate said spool thereby feeding said a segment of said sensor sheet into said sensing channel of said sensor cartridge.

17. The method of continuous measurement of activated clotting time as recited in claim 15 further comprising the steps of measuring the temperature of said blood aliquot and maintaining said blood aliquot at a desired temperature.
